(19) United States
(12) Patent Application Publication
(10) Pub. No.: US 2025/0332338 A1
(43) Pub. Date: Oct. 30, 2025

(54) DEVICES, SYSTEMS, AND METHODS FOR FLOW MANAGEMENT IN A FLUID MANAGEMENT SYSTEM

(71) Applicant: BOSTON SCIENTIFIC SCIMED, INC., Maple Grove, MN (US)

(72) Inventors: Cassandra Anne Hailson, Marlborough, MA (US); Damien Fitzgerald, Tipperary (IE); Teagan Jo Walter, Boston, MA (US); Troy Velazquez, Salem, NH (US); John O'Donnell, Chaska, MN (US); Randall Maas, Chaska, MN (US); Guilherme Militz, Tipperary (IE); Jessica Williams, Marlborough, MA (US); Anant Vaidyanathan Subramaniam, Boston, MA (US)

(73) Assignee: BOSTON SCIENTIFIC SCIMED, INC., Maple Grove, MN (US)

(21) Appl. No.: 19/191,154

(22) Filed: Apr. 28, 2025

Related U.S. Application Data
(60) Provisional application No. 63/640,056, filed on Apr. 29, 2024.

(51) Int. Cl. A61M 3/02; A61B 1/015

(52) U.S. Cl. CPC A61M 3/0258; A61B 1/015; A61M 3/0201; A61M 2205/3334; A61M 2205/3355; A61M 2205/3365; A61M 2205/502

(57) ABSTRACT

A fluid management system including a fluid management console and a fluid cassette configured to be received within the fluid management console. The fluid management console may comprise a housing, a controller housed within the housing, an inflow pump including a motor disposed within the housing, the inflow pump configured to provide fluid at a first setpoint, and a user input interface. The fluid cassette may be configured to provide a flow of fluid to a medical device. In response to a request to provide fluid in a flush mode at a second setpoint greater than the first setpoint, the controller of the fluid management console may be configured to operate the motor in a boost mode prior to reducing a motor speed to a flush mode speed.

### CROSS-REFERENCE TO RELATED APPLICATIONS

The application claims the benefit of U.S. Provisional Patent Application Ser. No. 63/640,056, filed on Apr. 29, 2024, the disclosure of which is incorporated herein by reference.

### TECHNICAL FIELD

The disclosure is directed to a fluid management system. More particularly, the disclosure is directed to flow management in a fluid management system.

### BACKGROUND

Flexible ureteroscopy (fURS), gynecology, and other endoscopic procedures require the circulation of fluid for several reasons. Surgeons today deliver the fluid in various ways such as, for example, by hanging a fluid bag and using gravity to deliver the fluid, filling a syringe and manually injecting the fluid or using a peristaltic pump to deliver fluid from a reservoir at a fixed pressure or flowrate via a fluid management system. Fluid management systems may adjust the flowrate and/or pressure at which fluid is delivered from the reservoir based on data collected from a procedural device, such as, but not limited to, an endoscope. Of the known medical devices, systems, and methods, each has certain advantages and disadvantages. There is an ongoing need to provide alternative medical devices and fluid delivery systems.

### BRIEF SUMMARY

This disclosure provides design, material, manufacturing method, and use alternatives for components of a fluid management system.

In a first example, a fluid management system may comprise a fluid management console and a fluid cassette. The fluid management console may comprise a housing, a controller housed within the housing, an inflow pump including a motor disposed within the housing, the inflow pump configured to provide fluid at a first setpoint, and a user input interface. The fluid cassette may be configured to be received within a receptacle of the housing of the fluid management console and to provide a flow of fluid to a medical device. In response to a request to provide fluid at a second setpoint greater than the first setpoint, the controller may be configured to operate the motor in a boost mode prior to reducing a motor speed.

Alternatively or additionally to any of the examples above, in another example, the boost mode may operate the motor at a speed greater than a speed required to deliver fluid at the second setpoint in a steady state.

Alternatively or additionally to any of the examples above, in another example, the boost mode may be configured to operate the motor at the greater of a speed of three times or more the motor speed in a flow mode or a minimum speed.

Alternatively or additionally to any of the examples above, in another example, the boost mode may be configured to operate the motor at a predetermined speed.

Alternatively or additionally to any of the examples above, in another example, the controller may be configured to operate the motor in the boost mode for a predetermined length of time.

Alternatively or additionally to any of the examples above, in another example, the boost mode may be configured to increase a pressure of a fluid pathway of a fluid management system.

Alternatively or additionally to any of the examples above, in another example, a magnitude of the boost mode may be based, at least in part, on one or more inputs of a resistance parameter of a fluid pathway of a fluid management system.

Alternatively or additionally to any of the examples above, in another example, a length of time of the boost mode may be based, at least in part, on one or more inputs of a resistance parameter of a fluid pathway of a fluid management system.

Alternatively or additionally to any of the examples above, in another example, after operating the motor to provide fluid at the second setpoint for a period of time, the controller may be configured to reverse a direction of the motor to operate the motor in a reverse mode.

Alternatively or additionally to any of the examples above, in another example, reversing the direction of the motor may be performed in response to a call for a flowrate decrease.

Alternatively or additionally to any of the examples above, in another example, the motor of the inflow pump may be operated in the reverse direction for a predetermined length of time or until a predetermined pressure is obtained.

Alternatively or additionally to any of the examples above, in another example, a magnitude and/or a length of time of the reverse mode may be based, at least in part, on one or more inputs of a resistance parameter of a fluid pathway of a fluid management system.

In another example, a method for controlling a fluid flow in a fluid management system may comprise operating a motor of an inflow pump at a first setpoint to provide fluid at a first flowrate, receiving a call for a flowrate increase to a second flowrate greater than the first flowrate, operating the motor of the inflow pump at a second setpoint to provide a third flowrate greater than the second flowrate, and after operating the motor of the inflow pump at the second setpoint for a predetermined length, operating the motor of the inflow pump at a third setpoint to provide fluid at the second flowrate.

Alternatively or additionally to any of the examples above, in another example, the method may further comprise after operating the motor of the inflow pump at the third setpoint for a length of time, reversing a direction of the motor and operating the motor of the inflow pump in the reverse direction.

Alternatively or additionally to any of the examples above, in another example, the motor of the inflow pump may be operated in the reverse direction for a predetermined length of time.

In another example, a method for controlling a fluid flow in a fluid management system may comprise operating a motor of an inflow pump in a flow mode for a first period of time, in response to a call for an increased flowrate, operating the motor in a boost mode for a second period of time, after the second period of time, operating the motor in a flush mode for a third period of time, and after the third period of time, operating the motor in a reverse mode for a fourth period of time.

Alternatively or additionally to any of the examples above, in another example, the boost mode may be configured to increase a pressure of a fluid pathway of a fluid management system.

Alternatively or additionally to any of the examples above, in another example, the reverse mode may be configured to decrease a pressure of a fluid pathway of a fluid management system.

Alternatively or additionally to any of the examples above, in another example, the flush mode may be configured to provide a fluid flowrate greater than a fluid flowrate of the flow mode and less than a fluid flowrate of the boost mode.

Alternatively or additionally to any of the examples above, in another example, a magnitude of the boost mode and/or the reverse mode may be based, at least in part, on one or more inputs of a resistance parameter of a fluid pathway of a fluid management system.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary console of a fluid management system;

FIG. 2 is a perspective view of a fluid management system including the console of FIG. 1 with a disposable fluid tubing set;

FIG. 3 is a perspective view of the front side of the fluid cassette of the disposable tubing set of FIG. 2;

FIG. 4 is perspective view of the rear side of the fluid cassette of the disposable fluid tubing set of FIG. 2;

FIG. 5 is a cross-sectional view of the fluid cassette of FIGS. 3 and 4 showing the internal fluid pathway therethrough;

FIG. 6 is an enlarged cross-sectional view of a portion of the fluid cassette of FIG. 5 showing the damping chamber;

FIG. 7A is a cross-sectional view of a portion of the fluid cassette of FIGS. 3 and 4 showing the internal fluid pathway with high inflow pump pressure; and

FIG. 7B is a cross-sectional view of a portion of the fluid cassette of FIGS. 3 and 4 showing the internal fluid pathway with low inflow pump pressure

FIG. 8 is an illustrative flow chart of a method for controlling the inflow pump; and

FIG. 9 is a schematic graph of the RPMs over time for a fluid management system delivering fluid at a first flowrate and a second flowrate using the illustrative method of FIG. 8.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

Some fluid management systems for use in flexible ureteroscopy (fURS) procedures (e.g., ureteroscopy, percutaneous nephrolithotomy (PCNL), benign prostatic hyperplasia (BPH), transurethral resection of the prostate (TURP), etc.), gynecology, and other endoscopic procedures may regulate body cavity pressure when used in conjunction with an endoscope device such as, but not limited to, a LithoVue™ Elite endoscope device using pressure and/or temperature data from the endoscope or other endoscopic device. Direct regulation of the intracavity pressure during a medical procedure may allow the fluid management system to safely drive system pressures of up to 600 mmHg to ensure no loss of flow during the procedure when tools are inserted into the working channel of the endoscope device. In some procedures, blood and/or debris may be present in the body cavity, which may negatively affect image quality through the endoscopic device. Fluid flow (e.g., irrigation) through the endoscopic device may be used to flush the body cavity to improve image quality. In some procedures, the body cavity may be relatively small and irrigation fluid may flow continuously. In some cases, it may be desirable to temporarily increase a flow of irrigation fluid. For example, a larger volume of fluid may be used to flush a body cavity. The responsiveness of the fluid management system may be countered by compliance in the fluid management system. For example, compliance in the fluid pathway may result in a smoother flow of fluid but may also result in a lower flow responsiveness. The present disclosure is directed towards a fluid management system which improves flow responsiveness without compromising the smoothness of the flow.

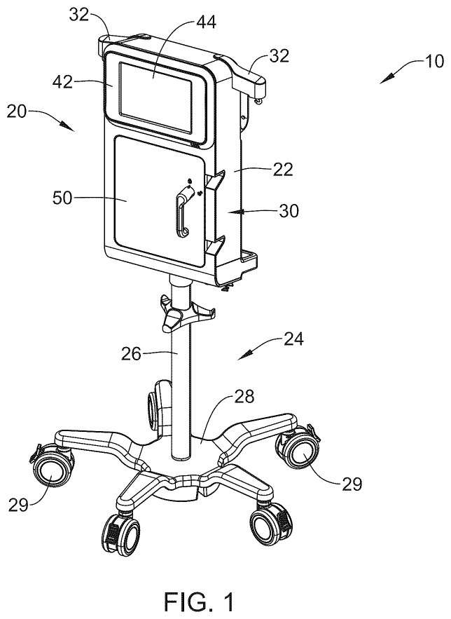

FIG. 1 is a schematic view of a fluid management system 10 that may be used in an endoscopic procedure, such as fURS procedures. The fluid management system 10 may be coupled to a medical device (not shown), such as an endoscope, that allows flow of fluid therethrough. As noted above, in some instances the endoscope may include a pressure sensor, such as the LithoVue™ Elite endoscope, or other endoscope. In some instances, the endoscope may include a temperature sensor to provide intracavity temperature feedback to the fluid management system 10, a pressure sensor to provide intracavity pressure feedback to the fluid management system 10, and/or a camera to provide visual feedback to the fluid management system 10.

The fluid management system 10 also includes a fluid management unit or console 20 including a controller 30 housed within a housing 22 of the console 20. In some instances, the console 20 may be portable and/or mobile such that the console 20 may be moved as desired. For instance, the console 20 may be mounted on a wheeled cart 24. For example, the wheeled cart 24 may include a pole 26 extending upward from a base 28. The base 28 may include a plurality of wheels 29 (e.g., caster wheels), allowing the cart 24 to be wheeled around to a desired location. In other instances, the console 20 may be provided with another form of cart, configured to be positioned on a flat surface, mounted to a wall, etc.

The fluid management system 10 may also include one or more user input interface components such as a touch screen interface 42. The touch screen interface 42 includes a display screen 44 and may include switches or knobs in addition to touch capabilities. In some embodiments, the controller 30 may include the touch screen interface 42 and/or the display screen 44. The user input interface, e.g., touch screen interface 42, allows the user to input/adjust various functions of the fluid management system 10 such as, for example flowrate, pressure, and/or temperature. The user may also configure parameters and alarms (such as, but not limited to, a max pressure alarm), information to be displayed, and the procedure mode. The user input interface, e.g., touch screen interface 42, allows the user to add, change, and/or discontinue the use of various modular systems within the fluid management system 10. The user input interface, e.g., touch screen interface 42, may also be used to change the fluid management system 10 between automatic and manual modes for various procedures. It is contemplated that other systems configured to receive user input may be used in place of or in addition to the touch screen interface 42 such as, but not limited to, voice commands.

The touch screen interface 42 may be configured to include selectable areas like buttons and/or may provide a functionality similar to physical buttons as would be understood by those skilled in the art. The display screen 44 may be configured to show icons related to modular systems and devices included in the fluid management system 10. The display screen 44 may also include a fluid flowrate and/or fluid pressure display. In some embodiments, operating parameters may be adjusted by touching a corresponding portion of the touch screen interface 42. The touch screen interface 42 may also display visual alerts and/or audio alarms if parameters (e.g., flowrate, temperature, etc.) are above or below predetermined thresholds and/or ranges. In some embodiments, the fluid management system 10 may also include further user interface components such as an optional foot pedal, a fluid warmer user interface, a fluid control interface, or other device to manually control various modular systems. For example, an optional foot pedal may be used to manually control flowrate. Some illustrative display screens 44 and other user interface components are described in commonly assigned U.S. Patent Application Publication No. 2018/0361055, titled AUTOMATED FLUID MANAGEMENT SYSTEM, the entire disclosure of which is hereby incorporated by reference.

The user input interface, e.g., touch screen interface 42, may be operatively connected to or a part of the controller 30. The controller 30 may be a CPU, including a computer, tablet computer, or other processing device. The controller 30 may be operatively connected to one or more system components such as, for example, an inflow pump, a fluid warming system, and a fluid deficit management system. In some embodiments, these features may be integrated into a single unit. The controller 30 is capable of and configured to perform various functions such as calculation, control, computation, display, etc. The controller 30 is also capable of tracking and storing data pertaining to the operations of the fluid management system 10 and each component thereof. In some embodiments, the controller 30 may include wired and/or wireless network communication capabilities, such as ethernet or Wi-Fi, through which the controller 30 may be connected to, for example, a local area network. The controller 30 may also receive signals from one or more of the sensors of the fluid management system 10. In some embodiments, the controller 30 may communicate with databases for best practice suggestions and the maintenance of patient records which may be displayed to the user on the display screen 44.

The fluid flowrate or the fluid pressure of fluid provided by the fluid management system 10 at any given time may be displayed on the display screen 44 to allow the operating room (OR) visibility for any changes. If the OR personnel notice a change in fluid flowrate or fluid pressure that is either too high or too low, the user may manually adjust the fluid flowrate or the fluid pressure back to a preferred level. The fluid management system 10 may also monitor and automatically adjust the fluid flowrate or the fluid pressure based on previously set parameters.

An illustrative fluid management unit may include one or more fluid container supports, such as fluid supply source hanger(s) 32, each of which may support a fluid supply source (e.g., fluid bag). In some embodiments, placement and/or weight of the fluid supply source(s) hanging from the fluid supply source hanger(s) 32 may be detected using a remote sensor and/or a supply load cell associated with and/or operatively coupled to each fluid supply source hanger 32 and/or fluid container support. The controller 30 may be in electronic communication with the supply load cell. The fluid supply source hanger(s) 32 may be configured to receive a variety of sizes of the first fluid supply source(s) such as, for example, 1 liter (L) to 5 L fluid bags (e.g., saline bags). It will be understood that any number of fluid supply sources may be used. The fluid supply source hanger(s) 32 may extend from the housing 22 of the console 20 and may include one or more hooks from which one or more fluid supply sources may be suspended. In some embodiments, the fluid used in the fluid management unit may be 0.9% saline. However, it will be understood that a variety of other fluids of varying viscosities, concentrations, mixtures, and/or consistencies may be used depending on the procedure.

In some embodiments, the fluid management unit may include one or more collection containers (not shown), for collecting waste fluid during a medical procedure. The collection containers (e.g., canisters) may be in fluid communication with a vacuum pump to provide suction for drawing fluid into the collection containers. The vacuum pump may be operatively and/or electronically connected to the controller 30. In some embodiments, the vacuum pump may be disposed within the fluid management system 10. Other configurations are also contemplated. In some embodiments, the collection container(s) may be operatively coupled to a collection load cell to detect placement and/or weight of fluid in the collection container(s) to contribute to a fluid deficit calculation.

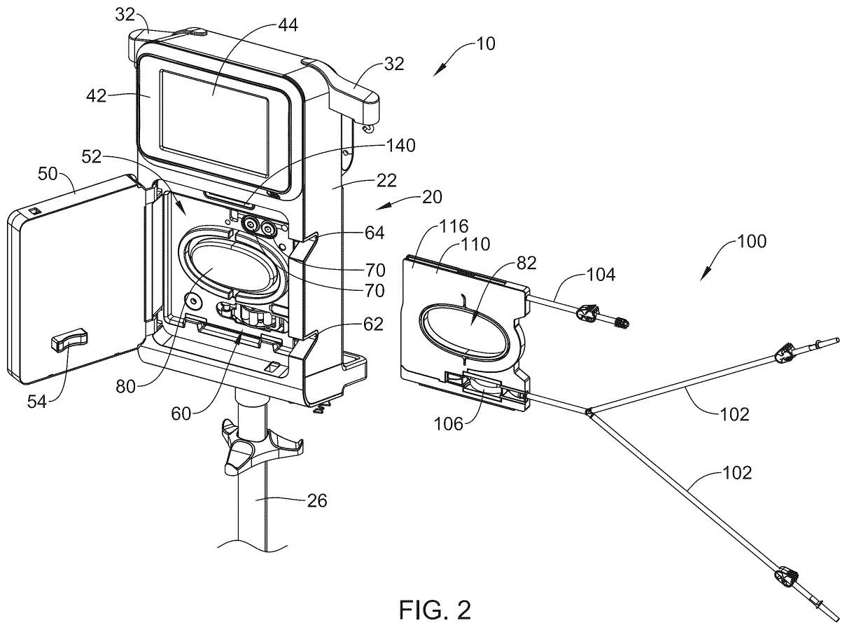

The console 20 may include a door 50 hingedly attached to the housing 22 of the console 20. As shown in FIG. 2, the door 50 may be opened to access a receptacle 52 configured to receive a fluid cassette 110 of a single use fluid tubing set 100 therein. The fluid management system 10 may include an inflow pump 60 configured to operatively engage the fluid tubing set 100 to pump and/or transfer fluid from a fluid supply source (e.g., a fluid bag, etc.) through the fluid tubing set 100 to a treatment site during a medical procedure. For example, the inflow pump 60 may be a roller pump or peristaltic pump positioned in the receptacle 52 configured to engage a length of flexible pump tubing 106 of the fluid cassette 110 when inserted therein. The door 50 may include an occlusion bed 54 mounted on the interior surface of the door 50. The occlusion bed 54 is configured to engage the length of flexible pump tubing 106 of the fluid cassette 110 when the door 50 is closed, to compress the length of flexible pump tubing 106 between the occlusion bed 54 and the inflow pump 60. The occlusion bed 54 may include a concave surface configured to engage the length of flexible pump tubing 106, which extends in an arcuate path around the inflow pump 60.

The inflow pump 60 may be electrically driven and may receive power from a line source such as a wall outlet, an external or internal electrical storage device such as a disposable or rechargeable battery, and/or an internal power supply. The inflow pump 60 may operate at any desired speed sufficient to deliver fluid at a desired pressure such as, for example, 5 mmHg to 50 mmHg, and/or at a target fluid flowrate or a target fluid pressure. The inflow pump 60 may be automatically adjusted based on, for example, pressure and/or temperature readings within the treatment site and/or visual feedback from the medical device attached thereto and inserted into the treatment site. In some embodiments, the controller 30 may be configured to control the inflow pump 60 to maintain a target or predetermined fluid flowrate or target fluid pressure based on a set of system operating parameters. In some embodiments, the controller 30 may be configured to control the inflow pump 60 to maintain a desired fluid pressure at the treatment site or a predetermined flowrate based on a set of system operating parameters.

The inflow pump 60 may also be manually adjusted via, for example, an optional foot pedal, the touch screen interface 42, voice commands, or a separate fluid controller. While not explicitly shown, the fluid controller may be a separate user interface including buttons that allow the user to increase or decrease the inflow pump 60. Alternatively, the fluid controller may be incorporated into the controller 30 and receive input via the touch screen interface 42, voice commands, or other means of input. It will be understood that any number of pumps may be used. In some embodiments, the fluid management system 10 may include multiple pumps having different flow capabilities. In some embodiments, a flow meter may be located before and/or after the inflow pump 60.

The fluid management system 10 may be user selectable between different modes based on the procedure, patient characteristics, etc. For example, different modes may include, but are not limited to, fURS Mode, BPH Mode, Hysteroscopy Mode, Cystoscopy Mode, etc. Once a mode has been selected by the user, mode parameters such as fluid flowrate, fluid pressure, fluid deficit, and temperature may be provided to the user via the display screen. The exemplary parameters of the specific modes may be previously determined and loaded onto the controller 30 using, for example, software. Thus, when a user selects a procedure from an initial display on the touch screen interface display screen 44, these known parameters may be loaded from the controller 30 to the various components of the fluid management system 10. The fluid management system 10 may also be user selectable between automatic and manual mode. For example, for certain procedures, the user may wish to manually adjust a fluid flowrate, fluid pressure, and/or other parameters. Once the user has selected the manual mode on, for example, the touch screen interface 42, the user may the adjust fluid flowrate or fluid pressure via other manual interfaces such as an optional foot pedal, voice commands, or the fluid control interface. If the user selects an automatic mode, the user may be prompted to select or input via the touch screen interface 42 which medical device (e.g., endoscope) is being used so that the controller 30 may determine if data obtained from the medical device can be used to facilitate control of the fluid management system 10. In some embodiments, the fluid management system 10 may be configured to verify the medical device (e.g., endoscope) selected is actually being used prior to using the collected data.

The single use tubing set 100 may include inflow tubing 102 providing a fluid inflow from the fluid supply source into the interior of the fluid cassette 110. In some instances, the inflow tubing 102 may include a bifurcated tubing with a first tubing section fluidly connected to a first fluid supply source and a second tubing section fluidly connected to a second fluid supply source. The first and second tubing sections may converge (such as at a Y-fitting) to a common tubing section extending to the fluid cassette 110. The end of the first tubing section and/or the second tubing section may include a bag spike, or other connector for connecting to the fluid supply source(s). The single use tubing set 100 may also include outflow tubing 104 providing a fluid outflow from the interior of the cassette 110 to a medical device connected thereto. The single use tubing set 100, including the fluid cassette 110, the inflow tubing 102, and the outflow tubing 104, may be disposable and provided sterile and ready to use.

When the fluid cassette 110 is installed in the receptacle 52 and the door 50 is closed, the inflow tubing 102 may pass through a channel 62 extending through a wall of the housing 22 of the console 20 to an exterior of the console 20. Likewise, when the fluid cassette 110 is installed in the receptacle 52 and the door 50 is closed, the outflow tubing 104 may pass through a channel 64 extending through a wall of the housing 22 of the console to an exterior of the console 20. The channel 62 and the channel 64 may both extend from the exterior of the console 20 to the receptacle 52. In some instances, both the channel 62 and the channel 64 may be located on the same sidewall of the console 20 such that both the inflow tubing 102 and the outflow tubing 104 extend from the console 20 on the same side of the console 20.

In some embodiments, the fluid management system 10 may include a fluid warming system 80, as shown in more detail in FIG. 2, for heating fluid to be delivered to the patient. The fluid warming system 80 may be an inductive heating system in some instances. In other instances, the fluid warming system 80 may be an infrared fluid warming system. Other fluid warming system configurations and methods may also be used, as desired. For example, the fluid warming system 80 may include one or more heat sources such as, for example a platen system or an inline coil in the fluid supply line to heat the fluid using electrical energy. Fluid warming may be specifically designed and tailored to the flowrates required in the specific application of the fluid management system 10. Some illustrative fluid warming systems are described in commonly assigned U.S. Patent Application Publication No. 2018/0361055, titled AUTOMATED FLUID MANAGEMENT SYSTEM, the entire disclosure of which is hereby incorporated by reference.

The fluid warming system 80 may include a heater configured to interact with the fluid cassette 110 to heat fluid passing therethrough. When the fluid cassette 110 is coupled with the heater, a susceptor positioned in the fluid path of the cassette 110 may be positioned within an induction coil of the fluid warming system 80 and be configured to heat the fluid flowing through or past the susceptor as the fluid passes through the fluid flow path of the cassette 110.

While not explicitly shown, the fluid warming system 80 may include a heater user interface included with or separate from the touch screen interface 42. In one example, the heater user interface may simply be a display screen providing a digital display of the temperature of the fluid entering and/or exiting the susceptor in the fluid flow path of the cassette 110. In another embodiment, the user interface may also include temperature adjustment buttons to increase or decrease the temperature of the fluid exiting the cassette 110. In this embodiment, the heater user interface and/or the display screen may indicate the current temperature of the fluid exiting the cassette 110 as well as the target temperature to be reached. It is noted that all information output from the fluid warming system 80 may be transmitted directly to the display screen 44 such that no heater user interface is necessary.

The fluid warming system 80 may include one or more sensors configured to monitor the fluid flowing therethrough. For example, temperature sensors may be mounted in the fluid warming system 80 such that they detect the temperature of the fluid flowing through the fluid cassette 110. In some embodiments, a first temperature sensor may be located at or near the fluid inlet to the susceptor and/or the fluid outlet from the susceptor so that they detect the temperature of fluid flowing through the fluid cassette 110 prior to the fluid entering the susceptor and after fluid exits the susceptor. In some embodiments, additional sensors may be located at a medial portion of the susceptor so that they detect a progression of temperature increase of the fluid in the fluid cassette 110.

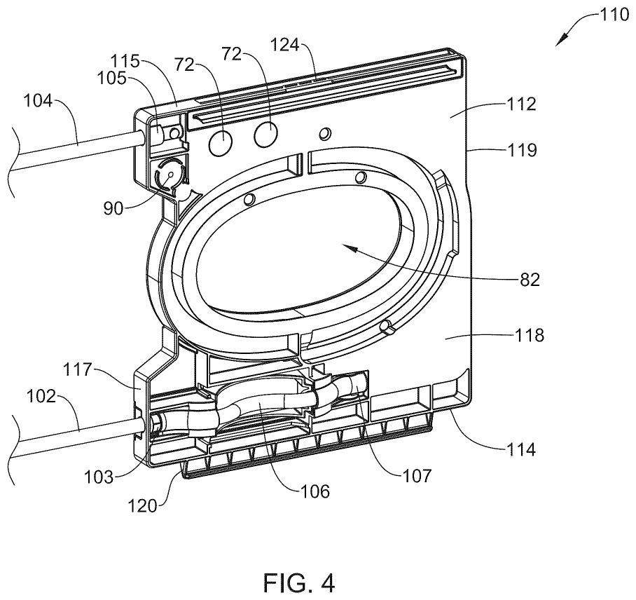

The console 20 may further include one or more additional sensors, such as a pressure sensor and/or a bubble sensor. For instance, the console 20 may include a pressure sensor 70, illustrated as a pair of pressure sensors, configured to monitor a system pressure of fluid exiting the cassette 110 and flowing through the outflow tubing 104 to a surgical site. The fluid cassette 110 may include a corresponding pressure sensor interface 72, such as a flexible membrane, (shown in FIG. 4) that allow the pressure sensor 70 to monitor the pressure of fluid flowing through the fluid cassette 110 when the fluid cassette 110 is installed in the receptacle 52 of the console 20. The pressure sensor 70 may send information to the controller 30 and/or display screen 44.

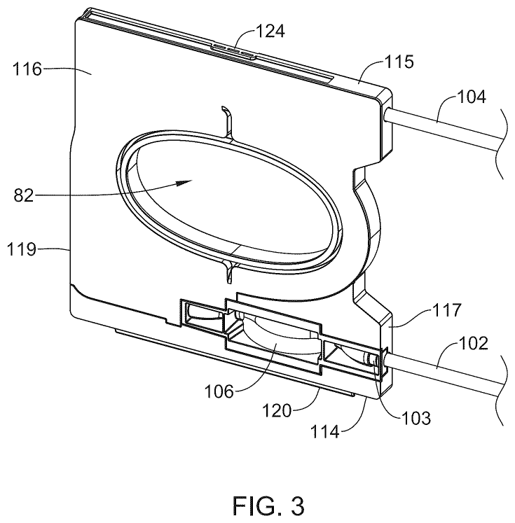

Additional features of the cassette 110 of the fluid tubing set 100 are illustrated in FIGS. 3 and 4. The fluid cassette 110 may include a housing 112 defining a fluid pathway through an interior of the housing 112. The fluid cassette 110 may include a front face 116 and a rear face 118 opposite the front face 116. The front face 116 is configured to face the door 50 of the console 20 when loaded in the receptacle 52, and the rear face 118 is configured to face a rear wall of the receptacle 52 of the console 20 when loaded in the receptacle 52. The fluid cassette 110 may also include an upper edge 115 and a lower edge 114 opposite the upper edge 115. The fluid cassette 110 may also include a first side edge 117 and a second side edge 119, opposite the first side edge 117. Both, the inflow tubing 102 and the outflow tubing 104 may extend from the first side edge 117. The housing 112 of the fluid cassette 110 may also include an opening 82, such as an oval opening, extending through the housing 112 from the front face 116 to the rear face 118. The opening 82 may extend a majority of the length of the housing 112 (i.e., a majority of the distance between the lateral edges of the housing 112) and/or a majority of the height of the housing 112 (i.e., a majority of the distance between the upper edge and the lower edge of the housing 112), in some instances. The opening 82 may be configured to receive an elevated portion of the rear wall of the receptacle 52, shown in FIG. 1 as the fluid warming system 80. The elevated portion of the rear wall of the receptacle 52 may be an oval shape sized to fit through the oval shaped opening 82 of the housing 112 of the fluid cassette 110 when the fluid cassette 110 is in its loaded position in the receptacle 52. In embodiments, in which the console 20 lacks a fluid warming system, the elevated portion of the rear wall of the receptacle 52 may still be present. Insertion of the elevated portion of the rear wall of the receptacle 52 through the opening 82 of the fluid cassette 110 may facilitate proper alignment of the fluid cassette 110 in the receptacle 52, for example.

In some embodiments, the fluid cassette 110 may include a fluid inlet port 103 and a fluid outlet port 105 located at a lateral side of the fluid cassette 110 accessible from the first side edge 117 of the fluid cassette 110. The fluid inlet port 103 may be coupled to the inflow tubing 102 and the fluid outlet port 105 may be coupled to the outflow tubing 104, with the inflow tubing 102 and the outflow tubing 104 extending laterally from the first side edge 117. The fluid inlet port 103 may be located below (e.g., closer to the lower edge 114) than the fluid outlet port 105. Thus, the inflow tubing 102 may extend laterally from the first side edge 117 at a location below (e.g., closer to the lower edge 114) than the location that the outflow tubing 104 extends from the first side edge 117. The cassette 110 may define an internal fluid pathway through an interior of the cassette housing 112 of the cassette 110 between the fluid inlet port 103 and the fluid outlet port 105. In embodiments of the fluid management system 10 including fluid warming capabilities, the internal fluid pathway may include the susceptor. The length of flexible pump tubing 106 of the cassette 110, configured to engage and be compressed by the rollers of the inflow pump 60, may extend from the fluid inlet port 103 to a connection 107 of the cassette 110 leading to the fluid pathway defined through the interior of the cassette 110. The flexible pump tubing 106 may be a discrete length of tubing separate from the inflow tubing 102 and the outflow tubing 104. In some instances, the flexible pump tubing 106 may extend through an arcuate pathway between the fluid inlet port 103 to the connection 107, such that the flexible pump tubing 106 follows the rotational path of the rollers of the inflow pump 60. The inlet port 103, the outlet port 105, and/or the connection 107 may be formed as a portion of the cassette housing 112 or may be formed separately and connected thereto.

The fluid cassette 110 may also include an air vent valve 90 configured to release air from the interior of the fluid cassette 110 to atmosphere. For example, the fluid cassette 110 may include an air vent including a hydrophobic membrane, allowing air, including bubbles entrained in the fluid, to pass through the hydrophobic membrane while preventing fluid within the fluid cassette 110 to pass therethrough. The air may then be vented to atmosphere through the air vent valve 90.

The fluid cassette 110 may also include one or more retention features configured to interact with the console 20 to retain the fluid cassette 110 in the receptacle 52 of the console 20. For example, the fluid cassette 110 may include a retention tab 120 extending from a lower edge of the housing 112 of the fluid cassette 110 and/or a retention tab 124 extending from an upper edge 115 of the housing 112 of the fluid cassette 110, configured to engage mating retention features of the console 20, as described in U.S. Provisional Application Ser. No. 63/597,481, entitled Fluid Management System With Disposable Fluid Cassette, filed on Nov. 9, 2023, the contents of which are hereby incorporated by reference in their entirety.

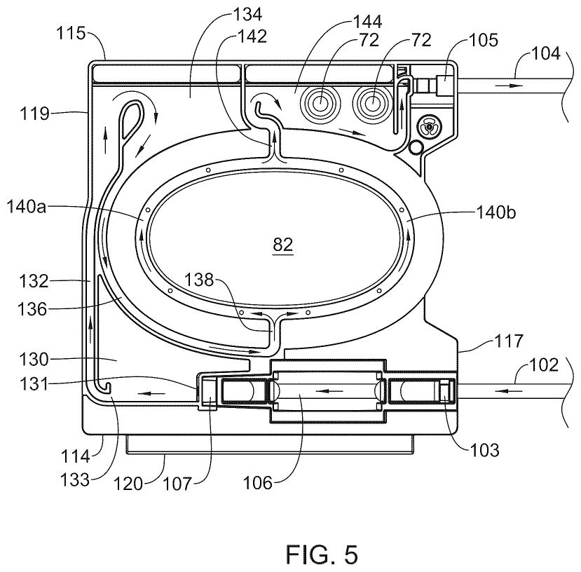

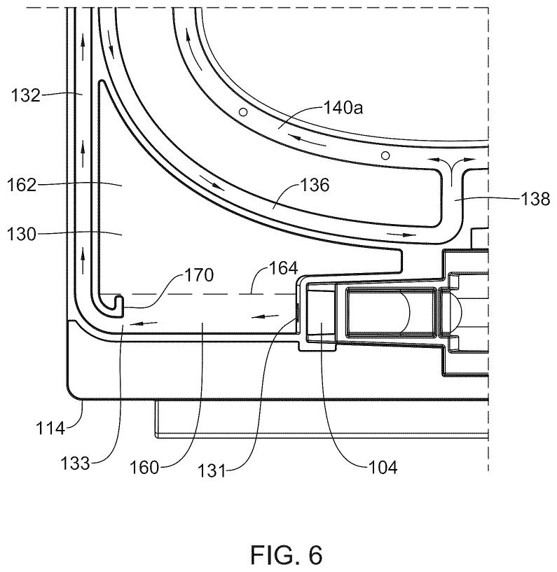

The internal flow pathway through the fluid cassette 110 is shown with arrows in the cross-sectional view of FIG. 5. Fluid flows into the interior of the fluid cassette 110 through the fluid inlet port 103 from the inflow tubing 102, and then passes through the pump tubing 106 as the pump tubing 106 is cyclically compressed by the rollers of the inflow pump 60. The fluid then flows into a fluid damping chamber 130 configured to reduce pressure fluctuations of the pulsatile fluid flow exiting the pump tubing 106 created by the inflow pump 60, and thus smoothen the fluid flow as the fluid exits the fluid damping chamber 130. The fluid damping chamber 130 may include a single fluid inlet 131 and a single fluid outlet 133. The single fluid inlet 131 and the single fluid outlet 133 may be located on opposite sides of the fluid damping chamber 130, such that fluid flows into the fluid damping chamber 130 through the fluid inlet 131 and flows out of the fluid damping chamber 130 through the fluid outlet 133. More details of the fluid damping chamber 130 will be described herein, in regard to FIG. 6.

As fluid exits the fluid damping chamber 130 through the fluid outlet 133 the fluid flows upward through the ascending fluid pathway 132. The ascending fluid pathway 132 interconnects the fluid damping chamber 130 with a first air vent chamber 134. The fluid then exits the first air vent chamber 134 in a downward direction along a descending fluid pathway 136. As shown in FIG. 5, the descending fluid pathway 136 may be an arcuate pathway extending from an upper region above the oval opening 82 to a lower region below the oval opening 82.

The fluid may then enter a bifurcated fluid pathway 140a/140b from the descending fluid pathway 136 as the fluid passes through a fluid warmer inlet channel 138 interconnecting the descending fluid pathway 136 and the bifurcated fluid pathway 140a/140b. The bifurcated fluid pathway 140a/140b includes a first fluid warming pathway 140a extending from the fluid warmer inlet channel 138 in a first direction and a second fluid warming pathway 140b extending from the fluid warmer inlet channel 138 in a second, generally opposite direction. The first fluid warming pathway 140a may extend around a first portion of the oval opening 82 on a first side of the oval opening 82 and the second fluid warming pathway 140b may extend around a second portion of the oval opening 82 on a second, opposite side of the oval opening 82. The bifurcated fluid pathway 140a/140b may then converge at a fluid mixing channel 142 located above the oval opening 82. Thus, the oval opening 82 may be located between the fluid mixing channel 142 and the fluid warmer inlet channel 138, such that the fluid mixing channel 142 is positioned above the oval opening 82 and the fluid warmer inlet channel 138 is positioned below the oval opening 82. Fluid may flow upward from the fluid mixing channel 142 into a second air vent chamber 144. The fluid may then exit the second air vent chamber 144 to the outflow tubing 104 through the outlet port 105.

FIG. 6 is an enlarged view of the portion of the fluid cassette 110 including the fluid damping chamber 130. During usage of the fluid cassette 110, a volume of fluid 160 may fill the lower portion of the fluid damping chamber 130 while a volume of air 162 is trapped in the upper portion of the fluid damping chamber 130. The fluid level 164 is the direct interface between the volume of fluid 160 and the volume of air 162. The fluid damping chamber 130 may be designed to substantially smoothen the pulsatile fluid flow from the inflow pump 60 for fluid flows up to 800 ml/min, in some instances. For example, it has been found that sizing the fluid damping chamber 130 such that the volume of air 162 is at least 38 ml substantially smoothens the pulsatile fluid prior to exiting the fluid damping chamber 130. Accordingly, the fluid damping chamber 130 may be sized to provide a volume of air 162 of 38 ml or more, or 40 ml or more, in some instances. For instance, the fluid damping chamber may be sized to provide a volume of air 162 of about 38 ml to 42 ml, during use.

Furthermore, as noted above, the fluid damping chamber 130 may include a single fluid inlet 131 and a single fluid outlet 133 located on opposite sides of the fluid damping chamber 130, such that fluid flows into the fluid damping chamber 130 through the fluid inlet 131 and flows out of the fluid damping chamber 130 through the fluid outlet 133. The fluid inlet 131 and the fluid outlet 133 may be positioned near a base of the fluid damping chamber 130. The fluid damping chamber 130 may be configured such that the upper extent of the fluid outlet 133 is lower (i.e., closer to the lower edge 114 of the fluid cassette 110) than the upper extent of the fluid inlet 131. This ensures that the fluid level 164 is above the upper extent of the fluid outlet 133 such that air from the volume of air 162 is not pulled out of the fluid damping chamber 130 into fluid exiting the fluid damping chamber 130 though the fluid outlet 133 into the ascending fluid pathway 132, which could otherwise occur at high flowrates. In some instances, the fluid outlet 133 may include a lip 170 extending upward from the upper extent of the opening of the fluid outlet 133 into the fluid damping chamber 130. The lip 170 may have any desired height. In some instances, the height of the lip 170 may be sized such that the fluid level 164 is above the upper extent of the lip 170. In other instances, the fluid level 164 may impinge the lip 170.

The fluid management system 10 may be operated in a pressure-controlled mode where the controller 30 increases and decreases the speed of the inflow pump 60 to achieve and maintain a pump pressure setpoint. The pump pressure may be related to the flowrate of fluid through the patient line, endoscope and the outflow path 104 as shown in Equation 1:

P=Q×R Equation 1

where P equals pump pressure, Q equals the flowrate of the fluid, and R equals the resistance. The resistance may be any restrictions to flow out of the fluid cassette 110. Assuming the resistance is constant the pump pressure and the flowrate increase proportionally. Thus, to increase the flowrate, the pump pressure is also increased.

In other examples, the fluid management system 10 may be operated in a flow-controlled mode where the controller operates the inflow pump 60 at a fixed speed and the pump pressure increases or decreases to maintain the desired or setpoint flowrate.

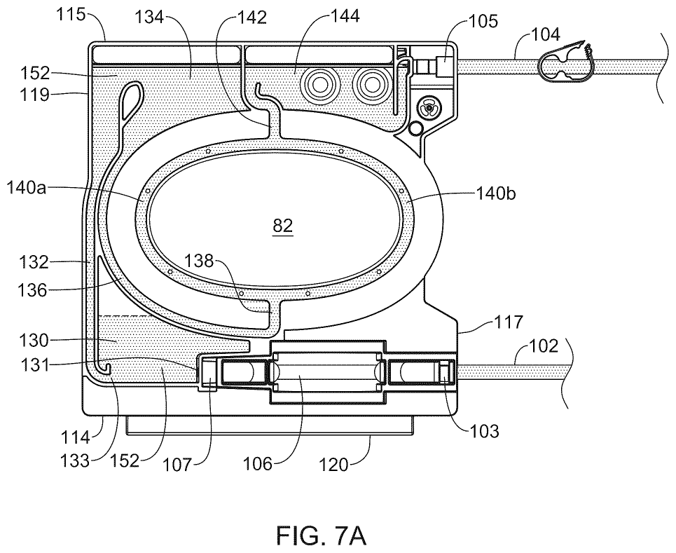

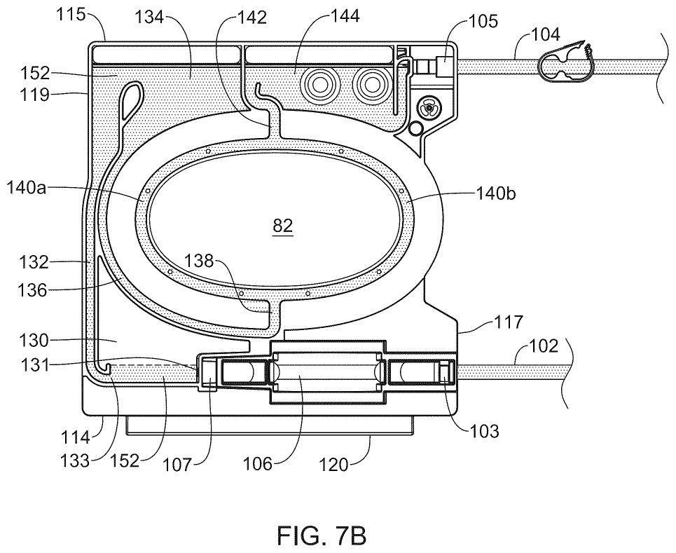

It may be desirable for the fluid management system 10 to deliver fluid smoothly and responsively. However, these two features may be counterproductive to one another. For example, compliance may be introduced into the fluid pathway to increase the smoothness of the flow. However, this may reduce system responsiveness. One illustrative source of compliance is the fluid damping chamber 130. As described herein, the fluid damping chamber 130 may include a volume of air. When the pump pressure increases, the volume of fluid 152 in the fluid damping chamber 130 increases compressing and pressurizing the trapped air, as shown in FIG. 7A which is a cross-sectional view of a portion of the fluid cassette of FIGS. 3 and 4 showing the internal fluid pathway with a higher pump pressure. As the pressure decreases, air expands and the fluid level in the fluid damping chamber 130 decreases, as shown in FIG. 7B which is a cross-sectional view of a portion of the fluid cassette of FIGS. 3 and 4 showing the internal fluid pathway with a lower pump pressure. Therefore, as the speed of the inflow pump 60 increases, pressure in the fluid cassette 110 (e.g., pump pressure) builds and some of the fluid goes into the fluid damping chamber 130 and compresses the air. The rest of the fluid travels through the remainder of the fluid pathway of the fluid cassette 110, into the patient line, and out of the tip of the endoscope. Once steady-state is reached (e.g., once the pump pressure reaches a constant value over time for a pressure-controlled mode or once the flowrate exiting the endoscope tip reaches a constant value over time for a flow-controlled mode), the amount of fluid being pushed by the inflow pump 60 is equal to the fluid being pushed out of the endoscope tip. This example demonstrates a physical representation of the relationship between pressure, flow, and volume of fluid stored due to compliance. Additionally, this illustrates that the speed of the inflow pump 60 does not immediately translate to flow speed through the patient line and endoscope. Said differently, the compliance in the fluid damping chamber 130 used to dampen flow oscillations introduces a delay in responsiveness.

In a fluid management system 10 having an inflow pump 60 when fluid flow is activated, if the controller 30 drives the motor of the inflow pump 60 at the revolutions per minute (RPM) representing the selected flowrate, it will take some time to build the pressure needed to drive that flowrate through the tip of the endoscope. For example, initially some fluid is diverted to the fluid damping chamber 130. Additionally, when the motor of the inflow pump 60 is reduced to zero RPM or the inflow pump 60 is deactivated (e.g., flow no longer desired), it will take time for the built-up pressure to dissipate or relieve itself through the tip of the endoscope. Each of these situations decreases the responsiveness of the fluid management system 10.

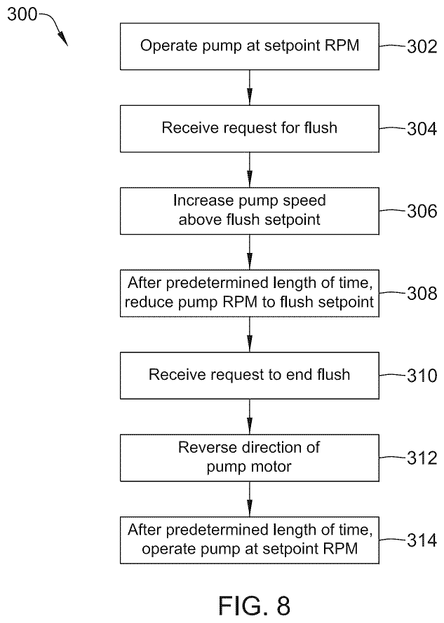

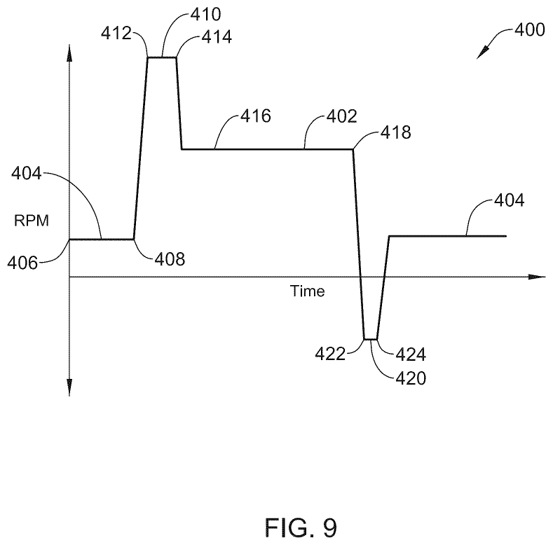

FIG. 8 is an illustrative flow chart 300 of a method for controlling the inflow pump 60 to increase responsiveness of the fluid management system while maintaining a smooth flow. For example, some procedures may require flowrates to quickly increase and/or decrease. In one example, a gradual change in the flowrate may not be sufficient to move a kidney stone. FIG. 9 is a schematic graph 400 of the RPMs or motor speed of an inflow pump 60 over time for a fluid management system 10 delivering fluid at a first or “flow” flowrate and a second or “flush” flowrate using the illustrative method of FIG. 8. To begin, the controller 30 may be configured to operate the inflow pump 60 at a first setpoint RPM, as shown at block 302 to provide fluid at a first flowrate. The first flowrate may be zero or greater than zero. The setpoint RPM may be determined based on a selected procedure or mode to provide a flow of fluid, if any, at a predetermined flowrate. The second or flush flowrate may be greater than the flow flowrate. The graph 400 may include an RPM or motor speed profile 402 for an illustrative portion of a procedure.

To begin, the inflow pump 60 is operated at a base or a first setpoint RPM or motor speed 404 from a first time point 406 to a second time point 408 to provide fluid at a first flowrate. In the illustrative example, at the first time point 406 the fluid management system 10 has already reached steady state for the flow flowrate. The first time point 406 does not necessarily represent the start of fluid flow through the fluid management system 10 and into the endoscope. The inflow pump 60 may be configured to provide a steady flow of fluid to the endoscope in a flow mode. At the second time point 408, the controller 30 may receive a call or a request for a flowrate increase, such as, but not limited to a call for a flush flowrate, as shown at block 304. The request may be received via a user input on a button of the endoscope (e.g., press a button), at the touch screen interface 42, or may be preprogrammed into the procedure mode. In other examples, the increase in flowrate may be based on the inflow pump pressure and/or intraluminal pressure headroom (as measured at a distal end of the endoscope). At the second time point 408, the speed of the pump motor is increased to a second speed 410 greater than the speed required to obtain the first, or flow, flowrate and greater than the speed required to obtain the second, or flush, flowrate. The motor of the inflow pump may be operated at the second speed 410 for a predetermined length of time (e.g., from a third time point 412 to a fourth time point 414). The time the motor of the inflow pump is operating at the second motor speed may be considered a boost phase or boost mode. In some examples, when the fluid management system 10 is transitioning to the boost mode to initiate a flush mode, the second motor speed may be a predetermined fixed RPM regardless of the speed the inflow pump 60 is operating at prior to the call for the flush flowrate. In one illustrative example, the second motor speed may be about 800 RPM. However, the second motor speed for the boost mode for transitioning to the flush mode may be less than 800 RPM or greater than 800 RPM, as desired. The boost phase or boost mode may be configured to quickly increase the pump pressure or a pressure of the fluid pathway of the fluid management system 10 to its intended steady state for the called for flowrate. Said differently, by increasing the speed of the motor of the inflow pump 60 above the called for setpoint, steady state of the called for flowrate may be obtained faster than if the motor of the inflow pump 60 is operated at the speed required to maintain steady state of the called for setpoint. It is contemplated that the resistance to flow out of the fluid cassette 110 may cause most of the fluid pushed during the boost phase 410 to enter the fluid damping chamber 130 such that a large bolus of fluid is not introduced into the connected device or endoscope. In some embodiments, the inflow pump 60 may be operated in the boost mode for a predetermined length of time. In one illustrative example, the inflow pump 60 may be operated in the boost mode for in the range of about 1 to about 4 seconds, about 1 to about 4.8 seconds, or about 1 to about 4.5 seconds. However, the duration may vary based on the flow setpoint, the boost setpoint, the flush setpoint, initial pump pressure, or the like.

After boosting the RPMs or the motor speed of the inflow pump 60 above the flush setpoint for a predetermined length of time, the controller 30 may lower the RPMs or motor speed of the inflow pump 60 to a third motor speed 416 for the flush flowrate, as shown at block 308. The third motor speed 416 may be greater than the first motor speed 404 and less than the second motor speed 410. The inflow pump 60 may be operated at the third speed 416 to operate at the flush flowrate for a period of time. When the inflow pump is operated at the third speed 416, fluid may be provided to the endoscope in a flush mode at the called for elevated fluid flowrate. At a fifth time point 418, the controller 30 may receive a call or a request to decrease the flowrate, end the flush flowrate, and/or return to the flow flowrate, as shown at block 310. In some cases, the request may be to terminate flow or stop the inflow pump 60 entirely. The request may be received via a user input on a button of the endoscope (e.g., press or release a button), at the touch screen interface 42, or may be preprogrammed into the procedure mode. For example, the flush flowrate, in which the RPMs of the motor increase to a predetermined higher motor speed (e.g., the third motor speed), may be active for a predetermined length of time programmed into the particular procedure mode. For example, a “low” flush may have a duration in the range of about 0.6 to about 1.3 seconds, a “medium” flush may have a duration greater than the duration of the “low” flush, such as in the range of about 1.3 to about 2.0 seconds, and a “high” flush duration may have duration greater than the duration of the “low” flush and the “medium” flush, such as in the range of about 2.0 seconds to about 3.6 seconds. These are just some examples. The flush durations may be less than or greater than the example ranges, as desired. In other examples, the flush flowrate may be determined by a proportional integral (PI) control. For example, the motor speed of the inflow pump 60 and the length of time the flush mode is active may vary according to pump pressure and/or control coefficients. In other words, the motor speed of the inflow pump 60 may be increased based on the current pump pressure when the flush mode is initiated and/or the duration of the flush mode may be based on the current pump pressure when the flush mode is initiated, in some instances. The motor speed of the inflow pump during the flush mode and/or the duration of the flush mode may be proportional to the pump pressure and/or based on control coefficients set in the controller in some instances.

At the fifth time point 418, or at the end of the flush flowrate, the motor of the inflow pump 60 may be reversed to relieve the elevated pump pressure, as shown at block 312. Reversing the motor of the inflow pump 60 may cause the motor to be operated in a reverse direction from the flow or flush modes to pull fluid out of the fluid damping chamber 130 to lower the pressure more quickly than simply returning the motor the RPMs necessary to push the flow flowrate (or another flowrate). The motor of the inflow pump 60 may be operated in the reverse direction 420 for a predetermined length of time (e.g., from a sixth time point 422 to a seventh time point 424) or until a predetermined parameter, such as, but not limited to, pump pressure, is reached. In an example, the motor of the inflow pump 60 may be operated in the reverse direction until the pump pressure reaches the pressure the system 10 was at when the flush was initially activated. In another examples, when flow is turned off after the flush mode, the motor of the inflow pump 60 may be operated in the reverse direction until the pump pressure drops to a predetermined pressure. In some examples, the predetermined pressure may be about 15 mmHg. However, the predetermined pressure may be less than 15 mmHg or greater than 15 mmHg, as desired. The time the pump motor is operating in reverse may be considered a reverse phase or reverse mode. The reverse phase or reverse mode may be configured to quickly decrease the pump pressure or a pressure of the fluid pathway of the fluid management system 10 to its intended steady state for the called for flowrate. After reversing the pump motor for a predetermined length of time or until another predetermined parameter is reached, the controller 30 may return the motor speed and direction of the inflow pump 60 to the first motor speed 404 and original setpoint for delivering fluid at the flow flowrate, as shown at block 314. This is just one example. In some cases, after the reverse phase, the pump motor may be turned off or deactivated. In some cases, the pump motor may be operated at a speed different from the first speed 404. It is contemplated that the speed may vary based on the selected procedure and/or desired outcome.

FIGS. 8 and 9 describe a boost mode for increasing the flowrate when a flush mode is called for. It is contemplated that a boost mode may also be used for starting fluid flow in the flow mode or anytime an increase in flowrate is called for. For example, when a procedure begins, the flowrate may be zero. When the controller 30 receives a call or request for fluid flow (for example, at the flow flowrate), the motor of the inflow pump 60 may be activated in a boost mode prior to operating at the speed required to maintain steady state at the flow flowrate. In some examples, the motor speed of the inflow pump 60 in the “flow” boost phase may be about three times greater than the setpoint speed for the flow mode. It is contemplated that the boost mode may be less than three times or greater than three times the setpoint speed of the flow mode. For example, in some cases, the boost mode may have a minimum motor speed of about 60 RPMs. In one illustrative example, if the motor speed setpoint for the flow mode is 50 RPMs, the motor speed for the boost mode may be 150 RPMs. In another example, if the motor speed setpoint for the flow mode is 12 RPMs, three times the flow setpoint (e.g., 3 times 12 equals 36) does not meet the example minimum RPMs (e.g., 60 RPMs) for the boost mode so the motor speed for the boost mode may set to the minimum allowable RPMs, or 60 RPMs in the case of the above minimum speed example.

The boost phase or boost mode may be configured to quickly increase the pump pressure or a pressure of the fluid pathway of the fluid management system 10 to its intended steady state for the called for flowrate. Said differently, by increasing the speed of the motor of the inflow pump 60 above the called for setpoint, steady state of the called for flowrate may be obtained faster than if the motor of the inflow pump 60 is operated at the speed required to maintain steady state of the called for setpoint. It is contemplated that the resistance to flow out of the fluid cassette 110 may cause most of the fluid pushed during the boost phase 410 to enter the fluid damping chamber 130 such that a large bolus of fluid is not introduced into the connected device or endoscope. In some embodiments, the inflow pump 60 may be operated in the boost mode for a predetermined length of time. In one illustrative example, the inflow pump 60 may be operated in the boost mode for in the range of about 1 to about 5 seconds, about 1 to about 4.8 seconds, or about 1 to about 4.5 seconds. However, the duration may vary based on the flow setpoint, the boost setpoint, the flush setpoint, initial pump pressure, or the like.

After boosting the RPMs or the motor speed of the inflow pump 60 above the flow setpoint for a predetermined length of time, or otherwise concluding the flush mode, the controller 30 may lower the RPMs or motor speed of the inflow pump 60 to a motor speed for the flow flowrate. It is contemplated when operating in the flow mode, the controller 30 may increase the flowrate for a flush mode or the flowrate may be decreased. When the flowrate is increased, boost modes may be used to increase the response time while maintaining a smooth flow. When the flowrate is decreased, the motor of the inflow pump 60 may be reversed to relieve the elevated pump pressure. Reversing the motor of the inflow pump 60 may cause the motor to be operated in a reverse direction from the flow or flush modes to pull fluid out of the fluid damping chamber 130 to lower the pressure more quickly than simply returning the motor the RPMs necessary to push the lower flowrate (or stop fluid flow). The motor of the inflow pump 60 may be operated in the reverse direction for a predetermined length of time or until a predetermined parameter, such as, but not limited to, pump pressure, is reached.

It is contemplated that the magnitude and/or length of the boost phase (for achieving flow flowrates, flush flowrates, or other flowrates) may vary depending on the procedure, anatomy, characteristics of the fluid management system 10, or the like. Similarly, the magnitude and/or length of the reverse phase may vary depending on the procedure, anatomy, characteristics of the fluid management system 10, or the like. In some embodiments, a mathematical model of the fluid management system 10 and/or one or more inputs of the resistance parameters may be used to calculate the boost necessary to achieve a desired flowrate within a predetermined length of time. For example, the resistance inputs may include a selection of a tool and/or sheath at the touch screen interface 42, a calibration and/or characterization step performed prior to the beginning of the procedure, data-driven detection, or the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A fluid management system, comprising:
a fluid management console, comprising:
a housing;
a controller housed within the housing;
an inflow pump including a motor disposed within the housing, the inflow pump configured to provide fluid at a first setpoint; and
a user input interface; and
a fluid cassette configured to be received within a receptacle of the housing of the fluid management console, the fluid cassette configured to provide a flow of fluid to a medical device;
wherein in response to a request to provide fluid at a second setpoint greater than the first setpoint, the controller is configured to operate the motor in a boost mode prior to reducing a motor speed.

2. The fluid management system of claim 1, wherein the boost mode operates the motor at a speed greater than a speed required to deliver fluid at the second setpoint in a steady state.

3. The fluid management system of any one of claim 1, wherein the boost mode is configured to operate the motor at the greater of a speed of three times or more the motor speed in a flow mode or a minimum speed.

4. The fluid management system of claim 1, wherein the boost mode is configured to operate the motor at a predetermined speed.

5. The fluid management system of claim 1, wherein the controller is configured to operate the motor in the boost mode for a predetermined length of time.

6. The fluid management system of claim 1, wherein the boost mode is configured to increase a pressure of a fluid pathway of a fluid management system.

7. The fluid management system of claim 1, wherein a magnitude of the boost mode is based, at least in part, on one or more inputs of a resistance parameter of a fluid pathway of a fluid management system.

8. The fluid management system of claim 1, wherein a length of time of the boost mode is based, at least in part, on one or more inputs of a resistance parameter of a fluid pathway of a fluid management system.

9. The fluid management system of claim 1, wherein after operating the motor to provide fluid at the second setpoint for a period of time, the controller is configured to reverse a direction of the motor to operate the motor in a reverse mode.

10. The fluid management system of claim 9, wherein reversing the direction of the motor is performed in response to a call for a flowrate decrease.

11. The fluid management system of claim 9, wherein the motor of the inflow pump is operated in the reverse direction for a predetermined length of time or until a predetermined pressure is obtained.

12. The fluid management system of claim 8, wherein a magnitude and/or a length of time of the reverse mode is based, at least in part, on one or more inputs of a resistance parameter of a fluid pathway of a fluid management system.

13. A method for controlling a fluid flow in a fluid management system, the method comprising:
operating a motor of an inflow pump at a first setpoint to provide fluid at a first flowrate;
receiving a call for a flowrate increase to a second flowrate greater than the first flowrate;
operating the motor of the inflow pump at a second setpoint to provide a third flowrate greater than the second flowrate; and
after operating the motor of the inflow pump at the second setpoint for a predetermined length, operating the motor of the inflow pump at a third setpoint to provide fluid at the second flowrate.

14. The method of claim 13, further comprising after operating the motor of the inflow pump at the third setpoint for a length of time, reversing a direction of the motor and operating the motor of the inflow pump in the reverse direction.

15. The method of claim 14, wherein the motor of the inflow pump is operated in the reverse direction for a predetermined length of time.

16. A method for controlling a fluid flow in a fluid management system, the method comprising:
operating a motor of an inflow pump in a flow mode for a first period of time;
in response to a call for an increased flowrate, operating the motor in a boost mode for a second period of time;
after the second period of time, operating the motor in a flush mode for a third period of time; and
after the third period of time, operating the motor in a reverse mode for a fourth period of time.

17. The method of claim 16, wherein the boost mode is configured to increase a pressure of a fluid pathway of a fluid management system.

18. The method of claim 16, wherein the reverse mode is configured to decrease a pressure of a fluid pathway of a fluid management system.

19. The method of claim 16, wherein the flush mode is configured to provide a fluid flowrate greater than a fluid flowrate of the flow mode and less than a fluid flowrate of the boost mode.

20. The method of claim 16, wherein a magnitude of the boost mode and/or the reverse mode is based, at least in part, on one or more inputs of a resistance parameter of a fluid pathway of a fluid management system.
